(19) United States
(12) Patent Application Publication
(10) Pub. No.: US 2026/0315017 A1
(43) Pub. Date: Oct. 15, 2026

(54) METHOD FOR CONTROLLING MOVEMENT AND MOBILE IMAGING DEVICE

(71) Applicant: Siemens Healthineers AG, Forchheim (DE)

(72) Inventors: Alexander Gemmel, Erlangen (DE); Hans-Peter Rieger, Walsdorf (DE); Martin Oßa Kafentzis, Heroldsberg (DE)

(21) Appl. No.: 19/642,588

(22) Filed: Apr. 8, 2026

(30) Foreign Application Priority Data
Apr. 9, 2025 (DE) 10 2025 113 993.8

(51) Int. Cl. A61B 6/00

(52) U.S. Cl. CPC A61B 6/4405; A61B 6/4441

(57) ABSTRACT

A method for controlling movement of a motorized mobile imaging device with a recording system that may be adjusted relative to an examination object, and an equipment trolley in an examination room includes providing a stored reference system-positioning configuration of the mobile imaging device relative to the examination object. The reference system-positioning configuration is suitable for recording an image of the examination object. The method includes estimating an environment model of at least part of the examination room based on at least the stored reference system-positioning configuration, determining a current system-positioning configuration, and determining a positional deviation in at least one degree of freedom between the current system-positioning configuration and the reference system-positioning configuration or between the current system-positioning configuration and the examination object estimated in the environment model. Movement of the imaging device is actuated. Movement behavior of the imaging device is executed depending on the positional deviation.

This application claims the benefit of German Patent Application No. DE 10 2025 113 993.8, filed on Apr. 9, 2025, which is hereby incorporated by reference in its entirety.

### BACKGROUND

The present embodiments relate to controlling movement in a mobile imaging device.

Mobile medical imaging devices (e.g., mobile C-arm X-ray devices) may have a mounting structure (e.g., a C-arm) with a recording system arranged thereon. The mounting structure is arranged on an equipment trolley. The mounting structure or the C-arm is equipped with drives in order, for example, to adjust the angle of inclination so that the recording system may be adjusted spatially in a plurality of projection directions. The C-arm itself may also be adjusted in different directions using axes. The equipment trolley is equipped with wheels on which the mobile imaging device may be moved manually. In addition, it is known to drive the wheels in a motorized manner to enable the equipment trolley to be moved with less effort or no effort and to automate certain movements of the mobile imaging device for clinical applications. Modern mobile C-arm X-ray devices are used in many areas (e.g., for intraoperative imaging during surgical procedures). In addition to simple projection images, modern mobile C-arm X-ray devices are also suitable to be used for 3D imaging or panoramic imaging. Such applications, for example, require the equipment trolley and the mounting structure to be positioned precisely and, in particular, repeatedly relative to the patient being treated before each application.

When treating patients in the operating room, mobile C-arm X-ray devices and patient tables are to be used in close proximity to one another. However, these are independent units that have no interface with one another. Therefore, the mobile C-arm X-ray device does not know the position of the patient table and the examination object (e.g., the patient). Movements of the C-arm X-ray device (e.g., both the equipment trolley itself and the recording system (C-arm)) for positioning and setting radiation parameters may only be performed generically or manually. This entails the risk of collisions between the mobile C-arm X-ray device and the patient table or the patient, and of inaccurate positioning, which results in poor image quality and delays due to the need for repeated repositioning.

Until now, only operators have been able to move the mobile C-arm X-ray devices in question to match the position of the patient table or to set radiation parameters appropriately for the position of the patient. Typical positions of the mobile image recording device may be stored, thereby enabling positions and orientations that have been set to be retrieved (semi)automatically.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a method that enables precise automatic positioning of a mobile imaging device relative to an examination object in a simple manner is provided. As another example, an imaging device suitable for performing the method is provided.

The method according to the present embodiments for controlling movement of a motorized mobile imaging device with a recording system that may be adjusted relative to an examination object and an equipment trolley in an examination room has the following acts: provision of at least one stored reference system-positioning configuration of the mobile imaging device relative to the examination object, where the reference system-positioning configuration is suitable for recording an image of the examination object; estimation of an environment model of at least part of the examination room, in particular, including the examination object, based on at least the stored reference system-positioning configuration; determination of the current system-positioning configuration; determination of a positional deviation in at least one degree of freedom between the current system-positioning configuration and the reference system-positioning configuration or between the current system-positioning configuration and the examination object estimated in the environment model; and actuation of a movement of the imaging device, where the movement behavior of the imaging device is executed in dependence on the positional deviation. The movement may have been planned in advance and may, but does not have to, be executed as a target in a system-positioning configuration already known to the imaging device for image recording.

The method enables quick and easy control of the movement of the imaging device with knowledge of at least one stored reference system-positioning configuration of the motorized mobile imaging device, and this results in the environment of the patient and the imaging device itself being safer and less prone to injury or damage. Herein, the stored reference system-positioning configuration is a system-positioning configuration in which an image of the examination object may be recorded or has already been recorded (e.g., such that the recording system irradiates the examination object or part of the examination object with X-ray radiation thereby providing that the image recorded is of suitable quality). The mere fact that an image may be recorded allows an estimate to be made of where the examination object (e.g., the patient and possibly also the patient table) is located. The method enables precise and safe automated movements of parts or components of the imaging device or the entire imaging device to be executed, thereby eliminating the need for additional time-consuming manual checks and thus reducing personnel costs. In addition, an environment model (e.g., a map of the examination object and possibly further objects in the examination room) is provided in a simple manner and without additional effort and may be used by the imaging device itself for automated movements. The environmental model may also be used by the operator in order, for example, to avoid collisions and thus enable safer movement of the device. The method is, for example, also suitable for motorized mobile imaging devices and self-propelled imaging devices.

For example, the movement behavior is actuated in dependence on the positional deviation with respect to at least one of the following (e.g., dynamic) parameters: motion dynamics of the equipment trolley and/or the recording system; speed of the equipment trolley and/or the recording system; acceleration or deceleration of the equipment trolley and/or the recording system; movement path of the equipment trolley and/or the recording system; movement direction of the equipment trolley; and/or the axes of the recording system. In one embodiment, a plurality of parameters or all parameters and/or other parameters not listed here are selected. Overall, the movement behavior may, for example, be such that high acceleration and/or speed is assumed in the event of a large positional deviation in at least one degree of freedom and low acceleration and/or speed in the event of a small positional deviation in at least one degree of freedom. For this purpose, it is, for example, also possible to use threshold values for the total deviation or for individual deviations of specific components to grade the parameters.

Further, it is also possible to preselect radiation parameters such as, for example, the X-ray dose or the SID distance in dependence on the positional deviation.

A system-positioning configuration may basically be understood to be the general position and orientation of all components of the imaging device. For example, in the case of an imaging device with an equipment trolley and an adjustable recording system, each system-positioning configuration may include an absolute position and absolute orientation of the equipment trolley of the imaging device and/or an absolute position and absolute orientation of the adjustable components of the recording system (e.g., the axes and the mounting structure; also relative to the equipment trolley). If the recording system is a typical C-arm mounting structure, the latter also includes, for example, the angulation of the C-arm of the C-arm X-ray device. Therefore, when determining the positional deviation in multiple degrees of freedom between, for example, the current system-positioning configuration and the reference system-positioning configuration, it is possible to determine both a deviation between the absolute position and orientation of the equipment trolley and a deviation between the absolute position and orientation of the mounting structures of the C-arm and a deviation of the angulation. Positional deviation in only one degree of freedom may, for example, be only the distance between the positions of the equipment trolley or the average distance of the imaging device.

According to one embodiment, the reference system-positioning configuration is optionally replaced by a new or updated reference system-positioning configuration. Therefore, the reference system-positioning configuration may, for example, be replaced by a further system-positioning configuration on the patient table/examination object that was adopted for image recording. The further system-positioning configuration is stored for this purpose. Alternatively, an updated reference system-positioning configuration may be calculated or estimated from a plurality of stored system-positioning configurations, or a reference system-positioning configuration may be simulated. Machine learning may also be used for this purpose. If an updated reference system-positioning configuration is determined, the updated reference system-positioning configuration replaces the original one in all steps (e.g., in the case of a repeated or further performance of the method), so that, for example, the positional deviation in at least one degree of freedom is then determined relative to the updated reference system-positioning configuration.

According to a further embodiment, further information (e.g., images and/or sensor data recorded by the imaging device in a system-positioning configuration) is used to optimize the estimation of the environment model. This enables the environment model to be improved and refined, thereby improving both the automatic movement control for the imaging device and the overview for the operator. Overall, the operation of the imaging device may be made safer. The sensor data may, for example, be data from cameras, proximity sensors, other collision sensors, or encoders in motorized axes or wheel drives.

According to a further embodiment, at least one further system-positioning configuration is stored and used to further optimize the estimation of the environment model. For example, if this, for example, also entails a system-positioning configuration that enables image recording, the position modeling of the examination object may be further refined. In addition, image information may be added here. However, it is also possible to use any system-positioning configuration to optimize the environment model. The mere fact that a system-positioning configuration is possible without a collision generates information about the arrangement of the environment and contributes to the environment model.

In one embodiment, images recorded by the imaging device in further system-positioning configurations are evaluated, and the evaluation is used to optimize the environment model. For example, the anatomy and position or orientation of the examination object may be modeled by analyzing, segmenting, and/or processing the image(s). Thus, a more accurate model of the environment (e.g., the arrangement of the examination object/patient and the patient table) is obtained with each further recording and storage position.

According to a further embodiment, a further current system-positioning configuration is determined, positional deviation in at least one degree of freedom between the further current system-positioning configuration and the reference system-positioning configuration or the further current system-positioning configuration and the examination object estimated in the environment model is determined, and the movement of the imaging device is actuated. The movement behavior of the imaging device is executed in dependence on the positional deviation in at least one degree of freedom. In this context, the method may be repeated as often as required with further current system-positioning configurations, and a corresponding iterative adaptation of the movement behavior may be performed. The environment model may also be refined in this way.

According to a further embodiment, the mobile imaging device is formed by an automated mobile C-arm X-ray device (e.g., a self-propelled mobile C-arm X-ray device). Such C-arm X-ray devices are known. Such C-arm X-ray devices have an equipment trolley with motorized wheels, and an X-ray source and an X-ray detector are arranged on the C-arm, which may be adjusted and rotated in at least three degrees of freedom.

According to a further embodiment, the previously determined environment model is displayed (e.g., on a monitor or a touchpad). It may also be provided that the operator verifies the environment model and corrects the environmental model or modifies the environmental model manually in the event of errors.

The present embodiments also include a motorized mobile imaging device for performing the method that includes: a motorized recording system that is adjustable relative to an examination object (e.g., a C-arm), with an X-ray source and an X-ray detector; a motorized equipment trolley with wheels; a computational unit (e.g., including one or more processors) for estimating an environment model of at least part of the examination room based on at least the stored reference system-positioning configuration; a memory unit (e.g., a memory) for storing a reference system-positioning configuration and further system-positioning configurations; a computational unit (e.g., including the one or more processors) for determining a positional deviation in at least one degree of freedom between system-positioning configurations and the reference system-positioning configuration or the examination object estimated in the environment model; and a control unit (e.g., a controller) for actuating the imaging device and for actuating the method.

### BRIEF DESCRIPTION OF THE DRAWINGS

FIG. 1 shows a sequence of acts of a method for controlling movement in an imaging device;

FIG. 2 shows a view of a mobile C-arm X-ray device for performing the method; and

FIG. 3 shows a view of examples of deviations between a reference system-positioning configuration and a current system-positioning configuration of a C-arm X-ray device.

### DETAILED DESCRIPTION

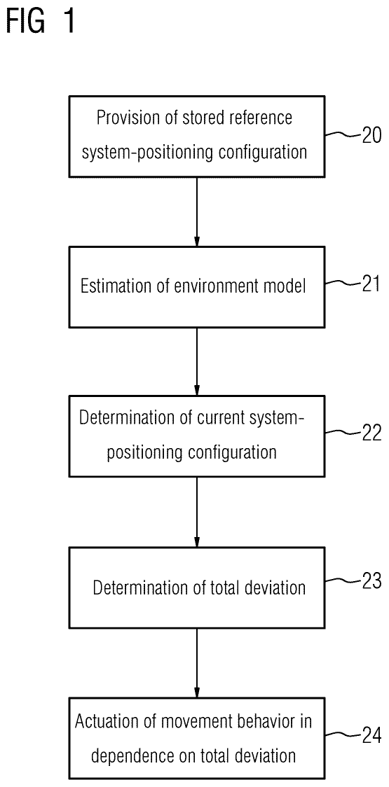

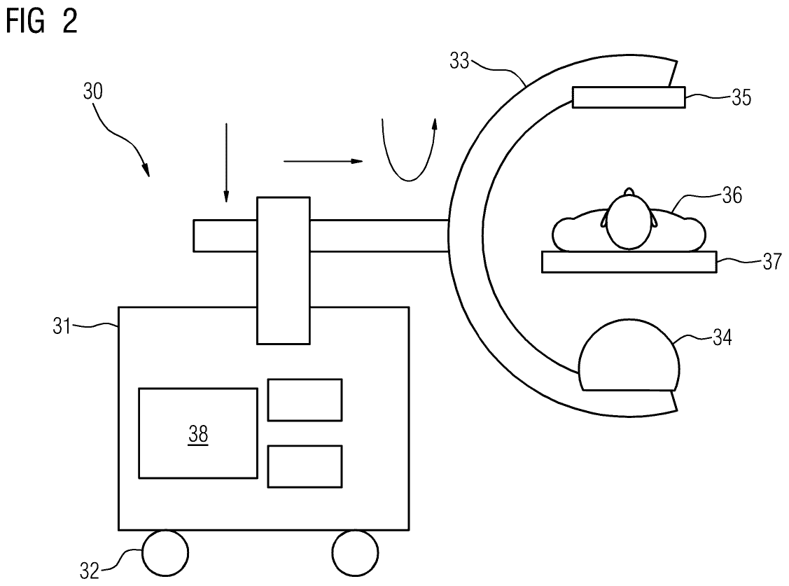

FIG. 1 lists steps of the method for controlling movement of an imaging device, which is, for example, formed by a mobile C-arm X-ray device 30 (see FIG. 2), where the mobile C-arm X-ray device 30 has a recording system that may be adjusted relative to an examination object 36 and a mobile equipment trolley 31 (e.g., automated mobile equipment trolley). The mobile C-arm X-ray device 30 may be located in an examination room in order to perform an X-ray examination of the examination object 36 (e.g., a patient). The patient may be arranged on a patient table 37. The mobile C-arm X-ray device 30 has a C-arm 33 on which an X-ray source 34 and an X-ray detector 35 are arranged. Herein, the C-arm 33 may be adjusted in multiple degrees of freedom. For example, the C-arm 33 may be translated in two or all three spatial directions by axes (see arrows in FIG. 2) and rotated about the examination object 36. The equipment trolley 31 may be moved on the floor of the examination room using wheels 32 that have a motorized drive and may be actuated. The mobile C-arm X-ray device 30 has a control unit 38 that is configured to actuate the motorized movements and to actuate the method. Further, the mobile C-arm X-ray device 30 has a memory unit 39 that is configured to store at least system-positioning configurations and the reference system-positioning configuration, and a processing unit that is configured to calculate total deviations between current system-positioning configurations and the reference system-positioning configuration.

In a first act 20 of the method for controlling movement of a motorized mobile C-arm X-ray device, at least one stored reference system-positioning configuration of the mobile imaging device relative to the examination object is provided. Herein, the reference system-positioning configuration may have been assumed previously and stored. A reference system-positioning configuration is a system-positioning configuration suitable for recording an image of the examination object (e.g., such that the equipment trolley and the C-arm are arranged in the vicinity of the patient table, the X-ray beam emitted by the X-ray source passes through the examination object, and the X-ray radiation thus modified strikes the X-ray detector). Storage may, for example, be provided in the memory unit 39 or in an external memory. Here, “provision” may be understood as providing that the reference system-positioning configuration is known to the C-arm X-ray device, is stored, and may be retrieved at any time and actuated if necessary.

As an example of a suitable C-arm X-ray device, system positions may, for example, be stored in the self-propelled mobile C-arm X-ray device CIARTIC Move from Siemens Healthineers AG; for example, it is possible to store a “table position” (e.g., a system-positioning configuration in which an examination object arranged on the patient table may be irradiated).

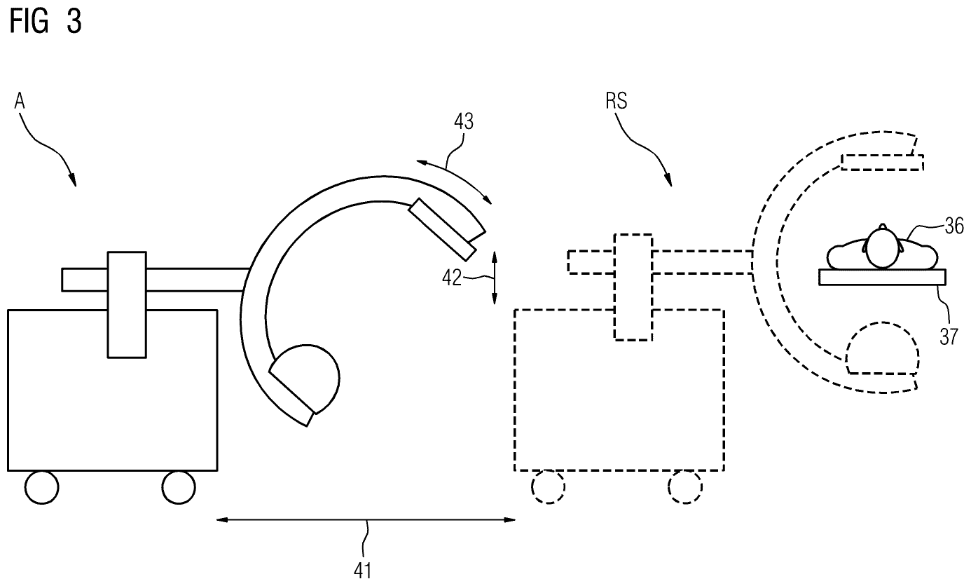

In FIG. 3, the dashed lines show an example of a reference system-positioning configuration RS of the mobile C-arm X-ray device 30 on a patient table 37 with an examination object 36.

In a second act 21, the stored reference system-positioning configuration is used to estimate an environment model of at least part of the environment. In the simplest case, the approximate location of the examination object is estimated, for example, midway between the X-ray source and the X-ray detector, since the reference system-positioning configuration is suitable for recording an image of the examination object. However, the environment model may be further refined with additional input, such as, for example, data from sensors such as, for example, cameras, proximity sensors, collision sensors, or encoders in motorized axes or drives. Images recorded in the reference system position may also be used to refine the environment model by applying image analysis with object recognition. Input from an operator may also be used to optimize the environment model (e.g., with regard to the size of the examination room or other objects and devices in the examination room).

Then, in a third act 22, the current system-positioning configuration of the C-arm X-ray device is determined (e.g., the general position of all components of the C-arm X-ray device at the current point in time). This may, for example, be performed by the control unit 38 of the C-arm X-ray device.

In a fourth act 23, a positional deviation in at least one degree of freedom between the current system-positioning configuration and the reference system-positioning configuration (e.g., alternatively, also, the examination object estimated in the environment model estimated) is then determined. Such a positional deviation may also have deviations in more than one degree of freedom (e.g., in multiple degrees of freedom), such as the deviation between the position and/or orientation of the equipment trolley, the deviation between the angulation of the C-arm, deviations in one or more axes of the mounting structure of the C-arm, deviations in orbital rotation, deviations in platform rotation, etc. FIG. 3 shows a positional deviation in three degrees of freedom between the current system position A and the reference system-positioning configuration of the C-arm X-ray device: the distance 41 resulting from translation of the equipment trolley (e.g., assuming that the spatial orientation remains unchanged), the height difference 42 resulting from upward movement of the C-arm, and the angular difference 43 resulting from rotation of the C-arm in the drawing plane. These are only examples; other degrees of freedom, such as, for example, distances, orientations, and angles may differ in other coordinates. In the event of a positional deviation in only one degree of freedom, the distance 41 of the equipment trolley may be used, for example.

In a fifth act 24, movement (e.g., at least one movement) of the imaging device is then actuated. The movement behavior of the imaging device is executed in dependence on the positional deviation. This may be any possible movement on the part of the imaging device or one or more components thereof, but above all, automatic movements without manual intervention (e.g., movement into a recording system-positioning configuration, into a parking system-positioning configuration, individual movements such as moving the equipment trolley to a different location, changes in angulation or combined movements). The movement may have been planned in advance (e.g., automatically) by the imaging device itself (e.g., via the control unit) or semi-automatically via target inputs from an operator.

The movement behavior may affect one or more of the parameters (e.g., dynamic parameters, such as the speed of the equipment trolley or other components (possible axis or C-arm), acceleration or deceleration of the equipment trolley or other components, movement paths or movement directions of the equipment trolley or other components). The dependence of the movement behavior on the positional deviation is, for example, such that short distances necessitate greater caution during movement, and, in contrast, longer distances enable limitations to be eliminated. This may, for example, take the following form: in the case of a large positional deviation in one or more or all degrees of freedom affected (e.g., a positional deviation above a first threshold value or above first threshold values (for different degrees of freedom)), all speeds and accelerations are possible (e.g., also high speeds) or are selected directly; in the event of a very small positional deviation in at least one degree of freedom (e.g., in the event of multiple degrees of freedom of the positional deviation, there may be limitations if the distance in one degree of freedom is small; below a second threshold value or below second threshold values (for different degrees of freedom)), high speeds and/or accelerations are blocked. The dependence may also be selected individually for each degree of freedom or each mobile component or axis, so that, for example, the speed of a component in one degree of freedom is limited by a short distance, while no such limit exists for a different component in another degree of freedom. It is also possible for multiple threshold values or a plurality of threshold values to be specified to grade the movement behavior in multiple stages or a plurality of stages.

It is also possible for a specific degree of freedom (e.g., the distance of the equipment trolley) to be determined and used as the limiting factor that predominantly determines the movement behavior.

It may also be provided that, in the event of a small total deviation, certain travel paths are blocked and/or other travel paths are given preference. It is also possible for certain directions of travel to be blocked based on the environment model that has already been created.

The determination of the current system-positioning configuration, determination of the positional deviation, and adaptation of the movement behavior may be repeated as often as desired during movement in order to perform the entire movement automatically and safely. For example, speed and acceleration may be reduced as the positional deviation decreases. Conversely, limitations may be lifted and speed increased as the distance from the reference system-positioning configuration increases.

Subsequently, all the data ascertained and (e.g., current at a given point in time) system positions and other sensor data ascertained may be used to refine the environment model. If, for example, one or more X-ray images of the examination object were taken after the imaging device had finished moving, the X-ray images may, for example, be analyzed by image or object recognition, and the data identified may also be used to optimize the environment model.

Subsequently, the reference system-positioning configuration may be replaced by a new or updated reference system-positioning configuration if necessary. The reference system-positioning configuration may, for example, be replaced by a further system-positioning configuration on the patient table/examination object that was assumed to record an image.

The latest self-propelled mobile C-arm X-ray devices offer users the option of storing system-positioning configurations (e.g., those used to record X-ray images; arranged on the patient table). Storing such a reference system-positioning configuration enables the relationship between the current system-positioning configuration (e.g., C-arm position, etc.) and the reference system-positioning configuration to be determined at any point from this time. The distance or the position of the C of the C-arm (e.g., larger orbital/angulation angle indicating positioning relative to the patient) may be used to indirectly infer whether the recording system is located in the vicinity of the patient. If the system is within a certain distance (e.g., a fine distinction may be made between platform rotation, longitudinal and lateral distance), the movement behavior of the system may be adapted. This applies, for example, to the dynamics (e.g., the selected speed, acceleration, and deceleration of the system axes), the travel paths (e.g., the way in which the system moves to a storage position), and the travel directions (e.g., mixed travel movements in lateral and longitudinal directions, or orbital and angulation directions may be blocked).

This system may be expanded to include any further system-positioning configurations from which an X-ray image was recorded, since recordings may only be taken on the table and on the patient. Image analysis may also be used to model the anatomy and patient position from the images. In this way, each further recording and storage position results in a more accurate environment model of the patient table and the position and orientation of the patient and the relationship thereto of the C-arm X-ray device. For example, the anatomy and position or orientation of the examination object may be modeled by analyzing, segmenting, and/or processing the image(s). In this way, each further recording and storage position results in a more accurate model of the table position and patient position. If necessary, the environment model may also be displayed on a monitor or another display unit. If necessary, the environment model may also be verified and/or corrected and/or overwritten by an operator. In addition, radiation parameters such as, for example, X-ray dose or SID distance may be preselected from the relationship between the reference system-positioning configuration and the current system-positioning configuration. The positional deviation from the reference system-positioning configuration may be used to determine whether the system-positioning configuration of the imaging device is currently further away from or closer to the table, and the dynamic parameters may be adapted accordingly.

Herein, a system-positioning configuration may be the combination of the position and orientation of the C-arm X-ray device in the examination room and the precise setting of the recording system with respect to its adjustable axes.

Independent of the grammatical term usage, individuals with male, female, or other gender identities are included within the term.

The present embodiments may be briefly summarized as follows. The present embodiments relate to a method for controlling movement of a motorized mobile imaging device with a recording system that may be adjusted relative to an examination object and an equipment trolley in an examination room with the following acts: provision of at least one stored reference system-positioning configuration of the mobile imaging device relative to the examination object, where the reference system-positioning configuration is suitable for recording an image of the examination object; estimation of an environment model of at least part of the examination room (e.g., including the examination object) based on at least the stored reference system-positioning configuration; determination of the current system-positioning configuration; determination of a positional deviation in at least one degree of freedom between the current system-positioning configuration and the reference system-positioning configuration or between the current system-positioning configuration and the examination object estimated in the environment model; and actuation of movement of the imaging device, where the movement behavior of the imaging device is executed in dependence on the positional deviation.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for controlling movement of a motorized mobile imaging device with a recording system that is adjustable relative to an examination object and an equipment trolley in an examination room, the method comprising:
providing at least one stored reference system-positioning configuration of the motorized mobile imaging device relative to the examination object, wherein the at least one stored reference system-positioning configuration is suitable for recording an image of the examination object;
estimating an environment model of at least part of the examination room including the examination object based on at least the at least one stored reference system-positioning configuration;
determining a current system-positioning configuration;
determining a positional deviation in at least one degree of freedom between the current system-positioning configuration and the at least one stored reference system-positioning configuration or between the current system-positioning configuration and the examination object estimated in the environment model; and
actuating movement of the motorized mobile imaging device, wherein a movement behavior of the motorized mobile imaging device is executed in dependence on the positional deviation.

2. The method of claim 1, wherein the movement behavior is executed in dependence on the positional deviation with respect to:
movement dynamics of the equipment trolley, the recording system, or the equipment trolley and the recording system;
speed of the equipment trolley, the recording system, or the equipment trolley and the recording system;
acceleration or deceleration of the equipment trolley, the recording system, or the equipment trolley and the recording system;
movement path of the equipment trolley, the recording system, or the equipment trolley and the recording system;
movement direction of the equipment trolley, axes of the recording system, or the equipment trolley and the axes of the recording system; or
any combination thereof.

3. The method of claim 1, further comprising replacing the at least one stored reference system-positioning configuration with a new or updated reference system-positioning configuration.

4. The method of claim 1, further comprising optimizing the estimation of the environment model using further information.

5. The method of claim 4, wherein the further information includes images, sensor data, or the images and the sensor data recorded by the motorized mobile imaging device in a system-positioning configuration.

6. The method of claim 5, wherein at least one further system-positioning configuration is stored and used to further optimize the estimation of the environment model.

7. The method of claim 1, wherein the positional deviation is a first positional deviation, and
wherein the method further comprises:
determining a further current system-positioning configuration;
determining positional deviation in at least one degree of freedom between the further current system-positioning configuration and the at least one stored reference system-positioning configuration or between the further current system-positioning configuration and the examination object estimated in the environment model; and
actuating the movement of the motorized mobile imaging device, wherein the movement behavior of the motorized mobile imaging device is executed in dependence on the positional deviation.

8. The method of claim 1, wherein the motorized mobile imaging device is formed by an automated mobile C-arm X-ray device.

9. The method of claim 1, wherein each system-positioning configuration comprises an absolute position and an absolute orientation of the equipment trolley of the motorized mobile imaging device, an absolute position and an absolute orientation of the recording system, or a combination thereof.

10. The method of claim 1, further comprising displaying the estimated environment model.

11. The method of claim 1, further comprising:
evaluating images recorded by the imaging device in system-positioning configurations; and
optimizing the environment model using the evaluating.

12. A motorized mobile imaging device comprising:
a motorized recording system that is adjustable relative to an examination object, the motorized recording system comprising an X-ray source and an X-ray detector;
a motorized equipment trolley with wheels;
a memory unit configured to store a reference system-positioning configuration and further system-positioning configurations;
one or more computational units configured to:
estimate an environment model of at least part of the examination room based on at least the stored reference system-positioning configuration; and
determine a positional deviation between system-positioning configurations and the reference system-positioning configuration or the examination object estimated in the environment model; and
a control unit configured to actuate movement of the motorized mobile imaging device,
wherein a movement behavior of the motorized mobile imaging device is executed in dependence on the positional deviation.
